(19) United States
(12) Patent Application Publication
(10) Pub. No.: US 2009/0088822 A1
(43) Pub. Date: Apr. 2, 2009

(54) THERAPEUTIC PULSE LASER METHODS AND APPARATUS

(75) Inventors: Ralph Pruitt, Aurora, CO (US); Gerry Graham, Centennial, CO (US)

Correspondence Address: SWANSON & BRATSCHUN, L.L.C., 8210 SOUTHPARK TERRACE, LITTLETON, CO, 80120, US

(73) Assignee: LED HEALING LIGHT, LLC, Aurora, CO (US)

(21) Appl. No.: 11/862,667

(22) Filed: Sep. 27, 2007

(51) Int. Cl. A61N 5/06

(52) U.S. Cl. 607/89

(57) ABSTRACT

A method and apparatus for providing therapeutic pulse laser treatment. The treatment method includes delivering more than one therapeutic laser light protocol from a hand held therapeutic laser device according to a preprogrammed schedule. At one or more select times during the preprogrammed schedule, the hand held therapeutic laser device will produce audio output. The audio output provides information to the user and directs him or her to execute a step in the overall treatment plan. For example, the audio output may signal to the user that it is necessary to make a change in or between treatment protocols, move the laser device, pause treatment, or otherwise control the delivery of therapeutic laser light. The ability of a relatively untrained user to implement the method may be enhanced if the audio output is delivered as synthetic speech output.

### TECHNICAL FIELD

The present invention is directed toward methods and apparatus for providing therapeutic pulse laser treatment and more particularly toward methods and apparatus for providing treatment at a remote location such as a patient's home.

### BACKGROUND ART

Light has a profound effect on the human body. Light therapies have proved beneficial in the areas of pain management and can further be used to specifically target individual pathogens or treat tissue dysfunctions or wounds. Light applied in a therapeutic manner can be either from a full or broad spectrum source or from a controlled source such as a laser which provides monochromatic light over a relatively narrow range of wavelengths.

Published U.S. Patent Application No. 2005/0245998, entitled “Hand Held Pulse Laser For Therapeutic Use” which is incorporated herein by reference in its entirety, discloses a hand held laser therapy device which features four laser sources providing pulsed laser light having a wavelength of about 635 nm to a patient. The device disclosed in the 2005/0245998 application has proved effective at treating numerous conditions and illnesses including but not limited to, RSD; head or other injuries; fibromyalgia; endocrine dysfunctions; low back and neck or other muscle/joint or skeletal pains. Laser therapy may also be used to treat allergies, infections and various diseases. In many instances, effective treatment requires multiple treatment sessions delivered over a period of hours or days.

The hand held therapeutic laser device of the 2005/0245998 application and similar devices can be relatively expensive. Known devices also require a certain degree of training to safely and effectively operate. Therapeutic use of a hand held pulse laser is somewhat simplified because various therapeutic treatment protocols may be stored in memory associated with the device. Thus, an operator may easily initiate the delivery of a therapeutic protocol by pressing an appropriate activation switch. The operator must, however, know where upon the patient's body to position and point the therapeutic laser for maximum effectiveness. In addition, certain treatable conditions require the delivery of multiple treatment protocols according to a structured schedule or treatment plan. Thus, a great deal of training is necessary before the operator has sufficient knowledge to assemble the necessary treatment protocols into an effective treatment plan. Accordingly, devices such as the hand held therapeutic laser fully disclosed in the 2005/0245998 application are not particularly well-suited for use by an untrained operator, for example, use by a patient at home in an unsupervised setting.

The present invention is directed toward overcoming one or more of the problems discussed above.

### SUMMARY OF THE INVENTION

One embodiment is a method of providing therapeutic pulse laser treatment. The treatment method includes delivering more than one therapeutic laser light protocol from a hand held therapeutic laser device according to a preprogrammed schedule. At one or more select times during the preprogrammed schedule, the hand held therapeutic laser device will produce audio output. The audio output provides information to the user and directs him or her to execute a step in the overall treatment plan. For example, the audio output may signal to the user that it is necessary to make a change in or between treatment protocols, move the laser device, pause treatment, or otherwise control the delivery of therapeutic laser light.

The use of audio output in the method allows a user with relatively less training to effectively deliver a treatment program which includes a complex treatment schedule, multiple therapeutic light protocols or the application of laser light at more than one position. Audio output may also be used to report the status of the delivery of a preprogrammed therapeutic schedule to the user.

The ability of a relatively untrained user to implement the method may be enhanced if the audio output is delivered as synthetic speech output. For example, various laser placement instructions may be delivered by synthetic speech output at appropriate times during a complex treatment schedule. Alternatively, device operation instructions, including but not limited to, instructions for the initiation, modification or termination of the delivery of one or more therapeutic protocols may be delivered from the laser to an untrained operator by synthetic speech output. A synthetic speech output enable device is thus well-suited for use by a patient at home.

Similarly, the effectiveness of the method of providing therapeutic pulse laser treatment may be enhanced by securing the laser in an operative position with a harness worn by a patient. Thus, the therapeutic pulse laser may be held in a location or position which is difficult for a home user to reach, for instance the center of a patient's back. Various types of harnesses may be implemented as articles of clothing allowing a home user to execute the treatment method while engaging in another activity such as watching television.

An alternative treatment method includes providing treatment at a remote location (away from caregiver's office) with a secondary pulse laser which may be, but does not have to be, less full-featured when compared to a master pulse laser. This treatment method includes programming the secondary hand held pulse laser with the master pulse laser or another device such as a computer maintained by the caregiver. The programming may include downloading one or more treatment protocols which may be grouped for delivery into complex treatment plans or schedules. A therapeutic protocol or treatment plan may be transmitted from the master pulse laser or other device to the secondary pulse laser over a wireless or wired communication link. The transmitted protocol or plans may be executed by the secondary pulse laser during remote operation, at a patient's home for example. The secondary pulse laser may produce audio output at select times during the execution of the protocol or plan indicating to the remote user that certain control steps or laser placement as described above are necessary.

Another embodiment is a hand held therapeutic pulse laser apparatus having an integrated audio output device and logic associated therewith configured to cause audio output upon the occurrence of select events such as described above in detail. The audio output device may include a synthetic speech output apparatus.

Another embodiment is a therapeutic pulse laser system including a master and secondary pulse laser. In this embodiment, a communication link between the master pulse laser and the secondary pulse laser may provide for downloading or transmission of one or more therapeutic protocols, treatment schedules or plans from the master pulse laser to the secondary pulse laser for remote execution with the secondary pulse laser. Thus, a skilled operator such as a physician may store an appropriate and relatively complex treatment schedule, plan or protocol into the master pulse laser unit. The physician may then transmit the treatment plan to a secondary hand held device which would be taken home by the patient. The patient may then execute the treatment plan with the secondary laser device at home. The secondary pulse laser may be full-featured or have limited control functionality. The effectiveness of remote treatment may be enhanced by providing the secondary laser device with audio output and in particular voice synthesis capabilities. In this manner, the secondary pulse laser may provide audible and understandable directions to the user for the delivery of an effective treatment regimen. The system may also include a harness for securing the therapeutic pulse laser in appropriate operative positions. The harness may be incorporated in or may be an article of clothing.

### BRIEF DESCRIPTION OF THE DRAWINGS

FIG. 1 is a plan view of a pulse laser for therapeutic use.

FIG. 2 is a plan view of a pulse laser for therapeutic use.

FIG. 3 is a plan view of a simplified pulse laser for therapeutic use.

FIG. 4 is a functional block diagram of a pulse laser for therapeutic use.

FIG. 5 is an exploded perspective view of a pulse laser for therapeutic use.

FIG. 6 is a plan view of a master and secondary pulse laser in a communication link configuration.

FIG. 7 is a flowchart illustrating a method disclosed herein.

FIG. 8 is a flowchart illustrating a method disclosed herein.

### DETAILED DESCRIPTION OF THE PREFERRED EMBODIMENT

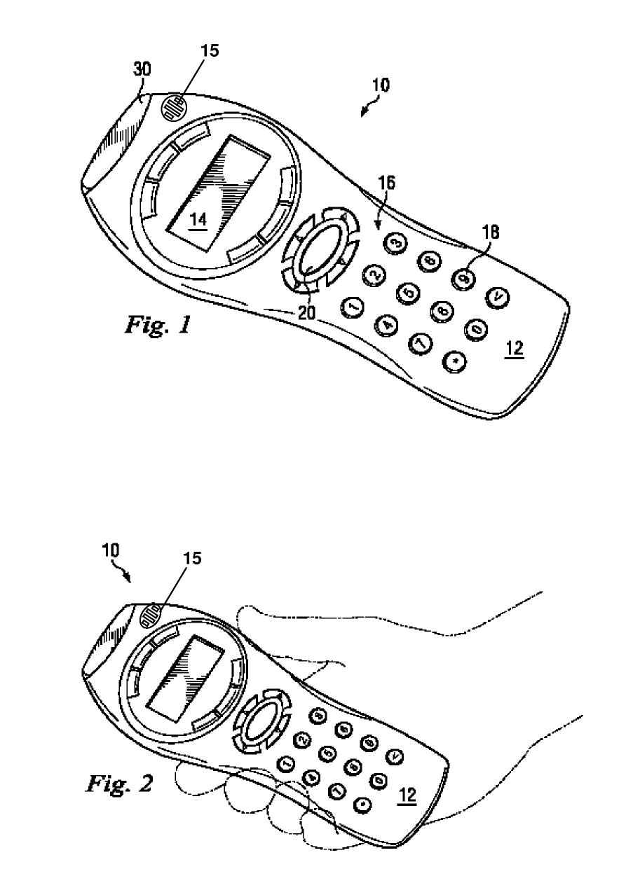

FIGS. 1 and 2 show a pulse laser for therapeutic use which is suitable for the implementation of the methods described herein. The full-featured hand held pulse laser of FIGS. 1 and 2 referred to herein as “pulse laser” 10 and includes various components contained within or on a housing 12. The housing 12 is sized to be comfortably held in the hand of an operator. A display panel 14 may be associated with the exterior of the housing 12. The display 14 can be used to display the operational status of the pulse laser 10 and can, in conjunction with an input keypad 16, be used to control the operation of the pulse laser 10.

The pulse laser 10 also includes an audio output. As described in detail below, the audio output may include a voice synthesis module in communication with an internal speaker or other acoustic transducer. As shown on FIGS. 1 and 2, the speaker may communicate with the exterior of the pulse laser 10 through a grill 15 or other opening in the housing 12.

Individual keys or buttons of the input keypad 16 can be associated with specific operation and control tasks. Representative examples of individual buttons used to control the operation of the pulse laser 10 include an on/off switch; a timer switch, useful for setting the duration of a pulse lasing treatment; and a light switch, used to backlight the display 14 for ease of visibility.

In addition, certain other input buttons of the input keypad 16 are preferably not associated with specific operational functions but are available to specifically program or set certain user designed or user accessed therapeutic lasing protocols to be executed by the pulse laser 10. In particular, scroll buttons, a cancel button, a select button, and delete button can all be used to maneuver through and select user operational and control menus displayed on the display 14. Alternatively, menu feedback and operational status information can be delivered audibly through the audio output. These buttons used in conjunction with a numeric keypad 18 can be used by an operator of the pulse laser 10 to select, modify, and deselect specific therapeutic protocols or regimens. The selected therapeutic protocols can be user designed, preprogrammed, manufactured, or downloaded to the pulse laser 10. The input keypad 16 may also include a laser pulse button 20 which allows an operator to manually pulse therapeutic laser light or initiate a selected therapeutic protocol.

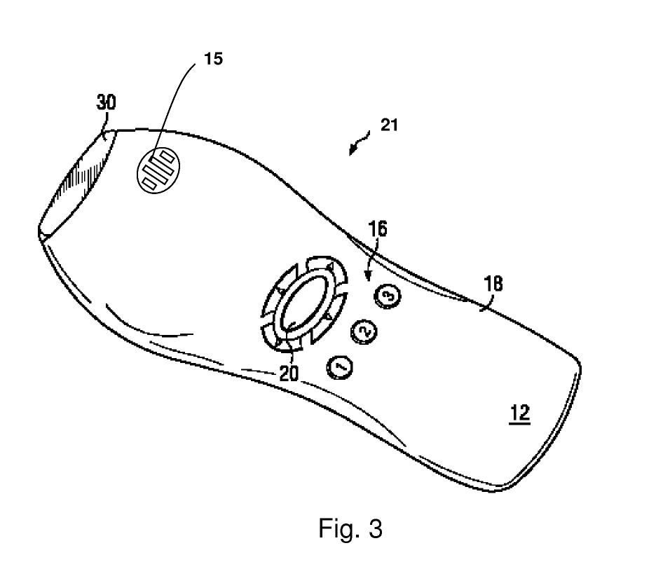

The specific nature or configuration of the input keypad 16 used to control and operate the pulse laser 10 can be varied. For example, a simplified hand held pulse laser unit 21 is shown in FIG. 3. The simplified pulse laser 21 is particularly well-suited for use at a remote location by a relatively unskilled operator, for example, use at home by the patient. The simplified pulse laser 21 may be a secondary pulse laser useful for implementing certain of the methods described below.

The simplified pulse laser 21 includes a housing 12 and simplified input keypad 16 which includes a laser pulse button 20. Also included in the simplified pulse laser 21 is an audio output which includes a grill 15 on the exterior surface of the housing 12. The simplified pulse laser 21 may be fabricated without a display to minimize costs.

The overall configuration of the housing 12 for either the pulse laser 10 or simplified pulse laser 21 is selected so that the entire pulse laser 10, 21 is self contained and is easily hand held, and the input keypad 16 is easily manipulated by the operator. Specific contours can be molded or otherwise fabricated into the housing 12 to achieve an ergonomically appropriate shape for hand held use.

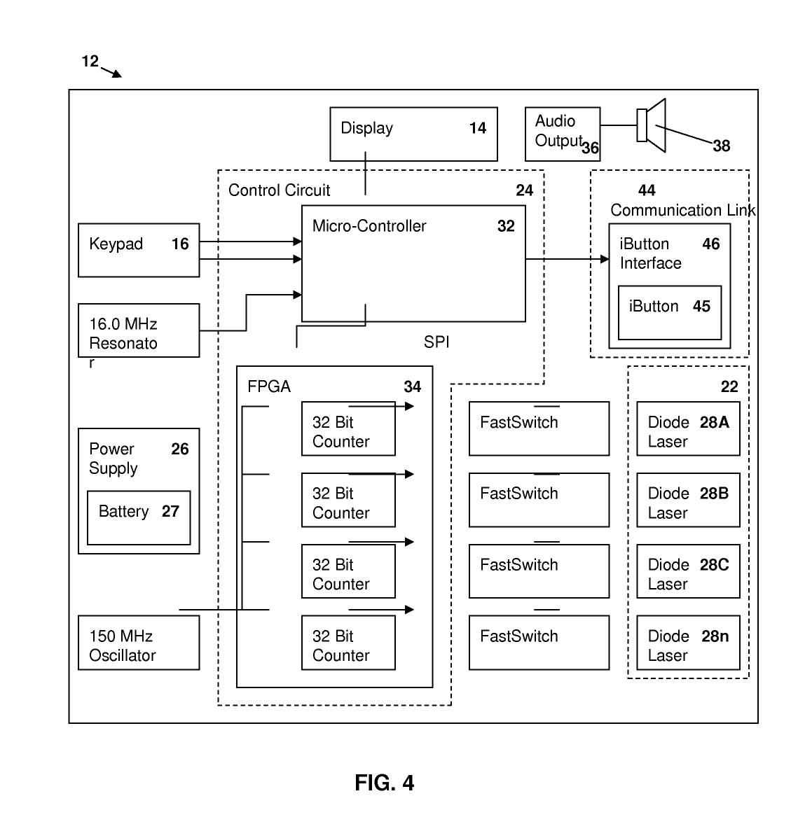

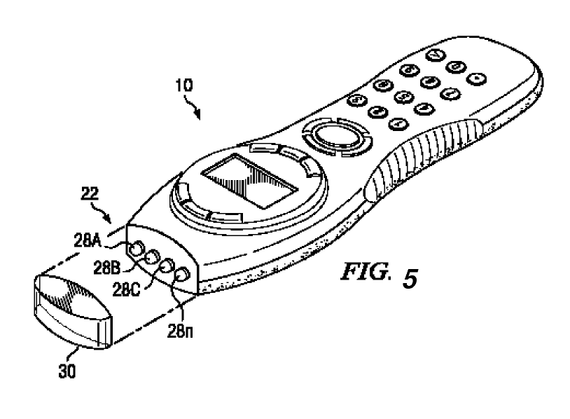

The pulse lasers 10, 21 include all components necessary for untethered operation within or on the housing 12. In particular, as shown in the block diagram of FIG. 4, a laser light source 22 and a control circuit 24 are operatively disposed on or within the housing 12. The control circuit 24 is in electronic communication with the laser light source 22 and configured to cause the laser light source 22 to emit pulsed laser light. Also included within the housing 12 is a power supply 26. Typically, the power supply 26 will be a battery 27 which may be a rechargeable battery such as a lithium ion battery, lithium polymer battery, or other type which is selected to provide a suitable voltage and amperage for operation of the control circuit 24, display 14, and laser light source 22, while being sized small enough to fit within the housing 12. Alternatively, the battery 27 may be a conventional non-rechargeable battery such as an alkaline or lithium battery. If the battery 27 is rechargeable, it is desirable that the battery 27 be readily and easily recharged thus minimizing unproductive time.

The laser light source 22 may include an array of diode lasers 28. The array as shown in FIG. 4 includes four diode lasers 28A, 28B, 28C . . . 28n, however, any suitable number of individual diode lasers 28 may be selected to form an array.

In the views of FIGS. 1-3, the laser light source 22, in particular the array of individual diode lasers 28A, 28B, 28C . . . 28n, is not visible as the laser light source 22 is positioned behind a guard 30 attached to the housing 12. In the exploded exterior perspective view of FIG. 5, the guard 30 has been removed and the laser light source 22, in particular an array of four diode lasers 28A, 28B, 28C . . . 28n is visible.

The geometric arrangement or focal direction of the diode lasers 28 included in the laser light source 22 can be selected to achieve specific therapeutic goals. Thus, the output from individual diode lasers 28A, 28B, 28C . . . 28n may be applied at different angles or different locations with respect to a treatment subject to achieve therapeutic goals. In addition, it is desirable that the control circuit 24 provide for the user selection of a suitable pulse rate from multiple possible pulse rates. Ideally, the individual diode lasers 28 of the laser light source 22 may be pulsed at multiple and independent user selectable pulse rates.

Also included within the housing 12 is a control circuit 24. In one embodiment of the therapeutic pulse lasers 10, 21 as depicted in FIG. 4, the control circuit 24 includes a microcontroller 32 in communication with a field programmable gate array 34 to generate a pulsed output signal to drive the laser array 22.

An audio output 36 and associated audio transducer 38 may also be included within the housing 12. The audio output 36 may be in communication with the microcontroller 32. The audio transducer 38 may be implemented with a small speaker or similar device. The audio output 36 may include circuitry which produces an amplified PWM (pulse width modulated) output signal. Such circuitry could be implemented with a Micro Analog Systems™ MAS 9118 analog interface or any similar part. An amplified PWM implementation would be suitable for producing a wide range of tones, beeps, buzzes, musical notes or other audio output suitable for conveying information to a user as described below. Alternatively, the audio output 36 could be implemented with a DAC (digital-analog-converter) with amplification or other suitable device capable of producing synthetic speech output. In a synthetic speech embodiment, various phrases, commands or instructions stored digitally in memory associated or communicating with the microcontroller 32 could be converted to an analog wave form by the audio output 36 resulting in synthetic speech being produced through the audio transducer 38.

The control circuit 24, and specifically the microcontroller 32, also receives user input from the input keypad 16 and outputs information to the display 14, audio output 36 or both if both are present n the device. It should be noted that the components depicted in FIG. 4 and described herein are one example of a suitable control circuit 24. Although this configuration is suitable for control of the output and functions of the therapeutic pulse lasers 10, 21 as described herein, other suitable circuits may be devised. The disclosed apparatus is not limited to the configuration depicted in FIG. 4.

The microcontroller 32 will preferably have programmable flash memory in addition to data processing circuitry. Many types of suitable onboard microcontrollers 32 are available commercially. For example, an ATmega32 or ATmega128 microcontroller by ATMEL Corporation is a suitable microcontroller for the control of the pulse lasers 10, 21. The apparatus described herein are not limited to these controllers, however. The disclosed apparatus may be implemented with any suitable control circuit.

Various therapeutic regimens can be programmed to the microcontroller 32 by use of the input keypad 16. However, manual programming can be time consuming and may result in an error. It is preferable to download treatment protocols, treatment plans or complex treatment schedules to the microcontroller 32 from a database associated with a separate apparatus. Accordingly, it is desirable to provide the pulse lasers 10, 21 with a communication link 44, to exchange information between the pulse laser and an external device such as a computer, database, or another full-featured pulse laser 10. Various types of suitable communication links 44 may be associated with the pulse lasers 10, 21 and contained within the housing 12 or located on the housing 12. For example, the communication link 44 may be removable storage media such as a memory stick, a miniature diskette or tape, or as is shown in FIG. 4, the apparatus for exchanging information 44 may be an iButton 45 communicating with an iButton interface 46 in communication with the microcontroller 32. Alternatively, the communication link 44 may be a wireless data transmitter operating with infrared, radio, or other wireless technology such as a Bluetooth® or similar link associated with the microcontroller 32. The apparatus for exchanging information could be as simple as a data port such as a USB, parallel, or serial port operatively associated with the housing 12 and communicating with the microcontroller 32. In such an implementation, the data port would be configured to receive a data cable for wired connection to an exterior computer, database, or second pulse laser 10.

The communication link 44 will provide for information to be downloaded to the pulse laser 10, 21, or for information to be uploaded from the pulse laser 10, 21 to a central database or another pulse laser 10, 21. For example, complicated treatment protocols or schedules may be downloaded from a central database to the pulse laser 10, 21. Similarly, treatment regimens developed by practitioners and found to be useful could be exchanged among practitioners over the internet. As described in detail below, a complicated treatment plan stored on a practitioner's master pulse laser 10 may be downloaded or transmitted to a secondary unit, which may be a simplified pulse laser 21, for remote use by the patient at home. In addition, updates to the functional capabilities or software of the pulse laser 10, 21 could be downloaded to the pulse laser 10, 21 through the communication link 44.

When the pulse laser 10, 21 is in use, power is supplied to the control circuit 24 and diode lasers 28 by an onboard power supply 26. Preferably, the power supply 26 will include a battery 27, typically a lithium ion, lithium polymer, or other type of battery 27 which can be quickly and repeatedly recharged. Preferably, the battery 27 can be removed from the housing 12 of the pulse laser 10 and swapped with a fresh battery 27 so that no down time is experienced if recharging becomes necessary while the pulse laser 10 is in use.

A full-featured hand held pulse laser unit such as pulse laser 10 can be relatively expensive to produce. In addition, a complex treatment regimen requires substantial expertise to deliver effectively. The methods described below enhance the ability of an untrained user, such as the patient himself or herself, to effectively deliver complex treatment plans remotely.

A treatment plan may include a group of distinct therapeutic laser light protocols which are each executed for a given period of time. A protocol as defined herein as a series of multiple laser modulation frequencies that are each executed for a given period of time. A macro is defined as a group of protocols. A macro may be a dedicated macro that is stored in non-volatile memory and which can not be programmed by the user or a user-programmable macro that can be fully input and modified by the user. Macros may be used to simplify the delivery of complex therapeutic plans. For example, certain treatable conditions require the delivery of ten or more protocols. The protocols may be grouped into macros, stored in memory and delivered automatically through memory associated with the hand held pulse laser 10, 21. Thus, it is no longer necessary for an operator to remember the sequence of multiple protocols with each having an individual treatment time.

A macro can be broken down into multiple steps. Each step includes the following sub-steps:

1. The protocol to be executed.

2. The amount of time to execute the protocol.

3. The delay time before executing the next protocol.

Many macros include separate protocols or sub-steps where laser light must be directed at different parts of the body at different points in time. A macro may require the pulse laser 10, 21 be turned on, off or otherwise controlled during the execution of the macro. Audio feedback generated by the audio output 36 through the audio transducer 38 greatly enhances the ability of an untrained operator to effectively deliver complex treatment. Audio output may allow the user to know the operational state of the pulse laser 10, 21 without visually viewing the unit. For example, the unit may beep or play a select tone at the completion of each protocol step which is included in a selected macro and similarly signal the initiation of the next step. Alternatively, if the pulse laser 10, 21 has speech synthesis functionality, the audio output may provide synthetic speech feedback and instructions. The unit may provide synthetic speech status reports during the execution of a complex macro. In addition, the pulse laser unit 10, 21 through the audio output 36 may provide instructions to the user directing the correct operation and delivery of a treatment regimen. For example, the audio output 36 may, through synthetic speech, tell the user where on the body to place the unit and where to point the unit. The unit through audio output 36 may provide basic operational instructions such as “depress laser pulse button for one minute”. Virtually any instruction regarding device operation or placement which is necessary to effectively deliver a treatment protocol or macro may be delivered to an untrained user through synthetic speech. Although the use of audio feedback to facilitate control of a pulse laser unit 10, 21 is described predominately with respect to synthetic speech feedback, it is important to note that the same goals may be accomplished with less sophisticated audio feedback such as beeps, tones, buzzes or other sound effects. A user of an embodiment which is not speech-enabled might have written instructions indicating that, for example, the unit is to be held against the patient's knee, and that the laser pulse button is to be depressed for one minute upon the playing of a specific tone. Select tones or other sound effects may be associated with specific operational tasks as well.

As described above, audio feedback facilitates use of a pulse laser 10, 21 by an untrained individual. Audio feedback also allows any user to know the operational state of the pulse laser 10, 21 without visually viewing the unit. Often, when the pulse laser 10, 21 is being used, it will be placed in positions where visual feedback shown on the display 14 is not readily seen or available.

In many instances, it may be difficult for a patient or other remote pulse laser user to effectively position a pulse laser 10, 21 when treating themselves. The methods and apparatus described herein may be implemented in conjunction with any type of clothing or harness that allows pulse lasers 10, 21 to be aimed at key joints or points of a human or animal for therapeutic treatment. Certain treatment protocols may require the laser to be held in position for 30 minutes or more. A macro may involve multiple protocols and treatments may extend over a period of several hours. A harness, specifically designed article of clothing or other type of support apparatus allows a user to effectively position a therapeutic pulse laser 10, 21 for effective hands-free treatment. The simplified pulse laser 21 may be produced as a relatively compact device and thus is particularly well-suited for implementation with a harness or article of clothing. A harness supported unit 10, 21 may be programmed to be left “on” for an extended period of time. The programming downloaded to the laser 10, 21 would contain instructions for the automatic execution of one or more treatment protocols at various times throughout the day without the unit being manually activated by the user. Thus, macros which require the delivery of various therapeutic protocols over an extended period of time may be automatically delivered without further input from the user and with minimal disruption of the patient's other activities.

The complex macros and therapeutic protocols which are components of an entire treatment schedule may be transmitted from one hand held master pulse laser 10 to a secondary pulse laser, for example, a simplified pulse laser unit 21. The transmission of a therapeutic protocol or macro from a master to a secondary pulse laser facilitates remote operation since it no longer is necessary to manually program the desired treatment plan into memory associated with the secondary unit. The communication link between master and secondary units may be implemented with any type of communication link known in the data processing arts. For example, a data cable, phone wire or other wired connection may be made between master and secondary units. The communication link may be made from the master unit to an intermediate device such as a personal computer, with a subsequent link being made from the intermediate device to the secondary unit for a two-step transmission of a therapeutic protocol from the master to the secondary.

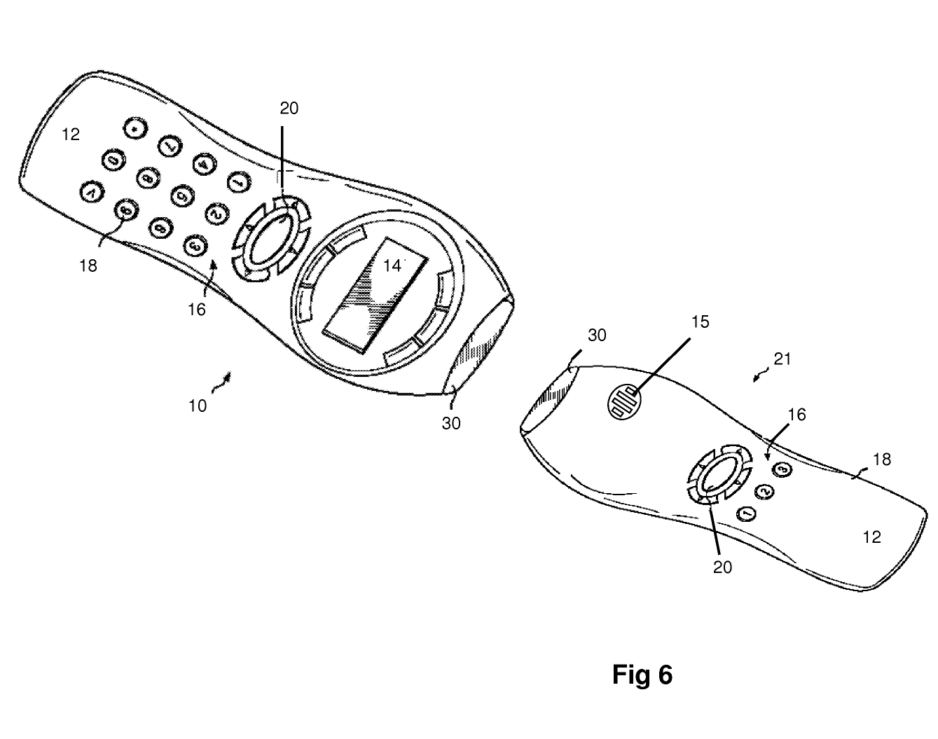

The communication link between the master pulse laser and secondary pulse laser may, as is shown in FIG. 6, be a wireless communication link. The wireless communication link may be implemented using a line of sight transmission protocol such as is commonly employed with television remote controls. Alternatively, a radio frequency link may be established using Bluetooth® or a similar protocol such that the master and secondary units need only be in relatively close proximity for a communication link and transmission to occur.

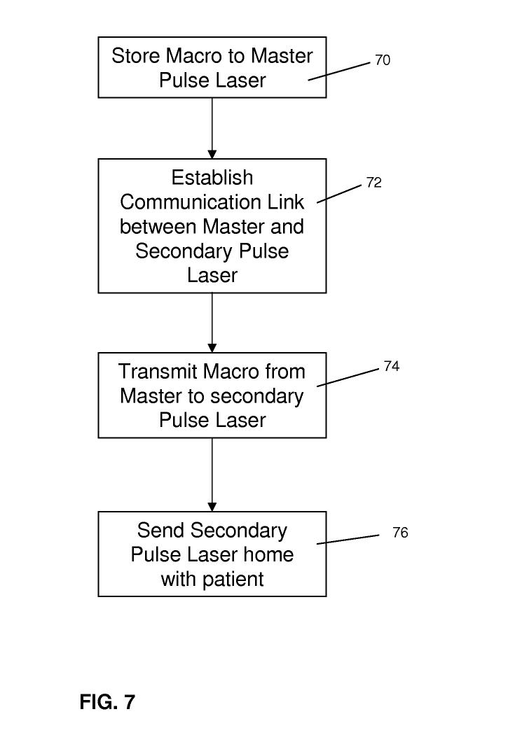

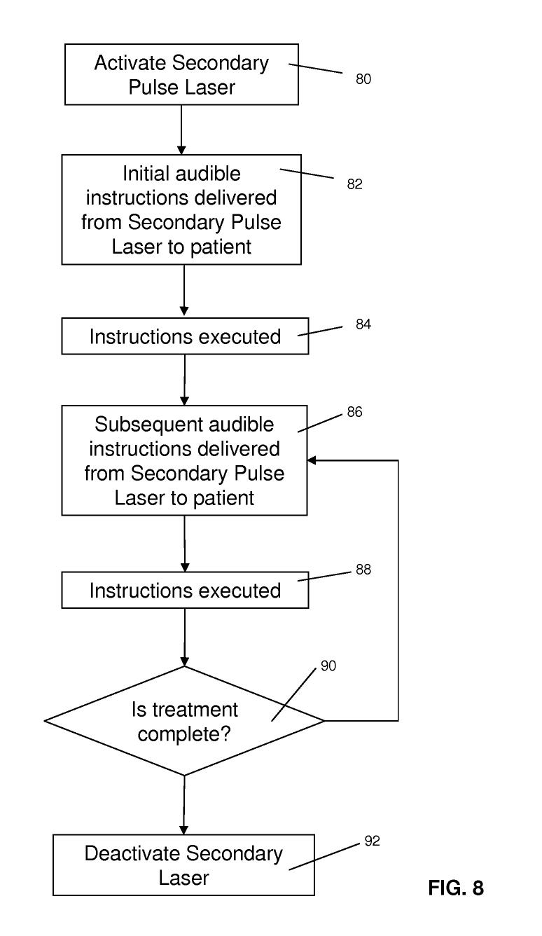

One non-limiting example of the methods described herein is graphically illustrated in the flowcharts of FIGS. 7 and 8. The method steps illustrated on FIG. 8 all take place at the work location of a physician, therapist, chiropractor or other primary caregiver. The method begins with step 70 where the primary caregiver stores at least one macro to a master pulse laser 10. As described above, a macro is a group of therapeutic protocols which, when executed, has been determined to provide therapeutic benefit. The individual protocols may be executed sequentially with or without periods of inactivity in between each protocol. Each protocol includes the delivery of therapeutic laser light from some or all of the output lasers 28 A-n which are modulated or pulsed at select rates for a pre-programmed duration. The macro(s) may be stored to the master pulse laser in step 70 by manually inputting the steps of the macro using the keyboard 16 associated with a master pulse laser 10. Alternatively, one or more macros may have been downloaded to the master pulse laser 10 from a computer, the internet, or from another pulse laser 10.

During the course of treatment, the primary caregiver may determine that a particular patient would benefit from remote therapy. Remote therapy may be indicated where therapy must be delivered over an extended period of time such as days or weeks. When the primary caregiver determines that remote therapy is appropriate, the primary caregiver may provide the patient with a secondary pulse laser which can be a simplified pulse laser 21, for home use. In step 72, the primary caregiver will establish a communication link between the master and secondary pulse lasers. After the establishment of a communication link, the macro(s) selected for treatment of the subject patient may be transmitted from the master pulse laser to the secondary pulse laser (step 74). The primary caregiver may then send the secondary pulse laser home with the patient (step 76).

The steps of the example method continue as shown on FIG. 8, a flowchart showing certain steps executed at the remote location, for example, a patient's home. When treatment is scheduled to commence, the patient may activate the secondary pulse laser (step 80). Upon activation, the secondary pulse laser will deliver audible instructions to the patient (step 82). As described in detail above, the audible instructions may be delivered through synthetic speech synthesis or the instructions may be delivered as non-speech sound effects such as buzzes, beeps or musical tones which have a predetermined meaning. For example, in a non-speech synthesis embodiment, it may be predetermined that a buzz tone delivered by the secondary pulse laser indicates that the patient is to place the pulse laser on a select body part and a ring tone indicates that the patient is to remove the pulse laser. Upon the receipt of audible instructions, the patient may execute the instructions (step 84). As described above, execution of the instructions may be facilitated by providing the patient with a specialized harness, sling or article of clothing which will hold the pulse laser 10, 21 in an operative position, allowing the patient to undertake other activities during treatment. At a select point in time dictated by the programming of the macro, subsequent audible instructions will be delivered from the secondary pulse laser to the patient (step 86). For example, the patient may, through synthesized speech or a predetermined sound effect, be instructed to move the pulse laser to another location on the body, depress one or more control keys or otherwise control the operation of the secondary pulse laser. Upon receipt of subsequent audible instructions, the patient will execute the instructions (step 88).

Subsequent instructions are audibly delivered and executed in this fashion until the treatment plan embodied in the macro is fully delivered (step 90). Upon completion of the pre-programmed macro, the patient may be audibly instructed to deactivate the secondary laser (step 92).

Those skilled in the art will recognize that the above example is not limiting upon the scope of the methods disclosed herein. The steps illustrated on FIGS. 7 and 8 may be executed in alternative orders and certain steps may be added or deleted without departing from the spirit and scope of the disclosed method.

The objects of the invention have been fully realized through the embodiments disclosed herein. Those skilled in the art will appreciate that the various aspects of the invention may be achieved through different embodiments without departing from the essential function of the invention. The particular embodiments are illustrative and not meant to limit the scope of the invention as set forth in the following claims.

1. A method of providing therapeutic pulse laser treatment comprising:
delivering more than one therapeutic laser light protocol from a hand held therapeutic laser device according to a pre-programmed schedule;
producing audio output at a select time during the pre-programmed schedule; and
controlling the delivery of a select therapeutic laser light protocol in response to the audio output.

2. The method of providing therapeutic pulse laser treatment of claim 1 further comprising:
tracking the status of the pre-programmed schedule; and
audibly reporting the status of the pre-programmed schedule to a user.

3. The method of providing therapeutic pulse laser treatment of claim 1 further comprising providing the audio output by synthetic speech output.

4. The method of providing therapeutic pulse laser treatment of claim 3 further comprising delivering therapeutic laser placement instructions from the laser to an operator by synthetic speech output.

5. The method of providing therapeutic pulse laser treatment of claim 3 further comprising delivering device operation instructions from the laser to an operator by synthetic speech output.

6. The method of providing therapeutic pulse laser treatment of claim 1 further comprising securing the laser in an operative position with a harness worn by a patient.

7. The method of providing therapeutic pulse laser treatment of claim 6 wherein the harness comprises an article of clothing.

8. A method of providing therapeutic pulse laser treatment comprising:
providing a handheld master pulse laser;
providing a handheld secondary pulse laser;
providing a communication link between the master pulse laser and the secondary pulse laser;
transmitting a therapeutic protocol from the master pulse laser to the secondary pulse laser for execution by the secondary pulse laser during remote operation; and
producing audio output at a select time during the execution of the therapeutic protocol; and
controlling the delivery of therapeutic protocol by the secondary pulse laser in response to the audio output.

9. The method of providing therapeutic pulse laser treatment of claim 8 further comprising:
tracking the status of the delivery of the therapeutic protocol; and
audibly reporting the status of the pre-programmed schedule to a user.

10. The method of providing therapeutic pulse laser treatment of claim 8 wherein the audio output comprises synthetic speech output.

11. The method of providing therapeutic pulse laser treatment of claim 10 further comprising delivering therapeutic laser placement instructions from the secondary pulse laser to an operator by synthetic speech output.

12. The method of providing therapeutic pulse laser treatment of claim 10 further comprising delivering device operation instructions from the secondary pulse laser to an operator by synthetic speech output.

13. The method of providing therapeutic pulse laser treatment of claim 8 further comprising securing the secondary pulse laser in an operative position with a harness worn by a patient.

14. A handheld therapeutic pulse laser comprising:
an audio output device;
a logic circuit receiving input from the pulse laser apparatus and in electronic communication with the audio output device, the logic circuit being configured to cause the audio output device to provide audio output upon the occurrence of select events.

15. The handheld therapeutic pulse laser of claim 14 wherein the audio output comprises synthetic speech output.

16. The handheld therapeutic pulse laser of claim 14 further comprising:
means for delivering more than one therapeutic laser light protocol from the hand held therapeutic laser device according to a pre-programmed schedule; and
means for controlling the delivery of a select therapeutic laser light protocol in response to the audio output.

17. The handheld therapeutic pulse laser of claim 16 further comprising:
means for tracking the status of the pre-programmed schedule; and
means for audibly reporting the status of the pre-programmed schedule to a user.

18. The handheld therapeutic pulse laser of claim 14 further comprising means for delivering therapeutic laser placement instructions from the laser to an operator by synthetic speech output.

19. The handheld therapeutic pulse laser of claim 14 further comprising means for delivering device operation instructions from the laser to an operator by synthetic speech output.

20. The handheld therapeutic pulse laser of claim 19 further comprising a harness for securing the therapeutic pulse laser in an operative position.

21. A therapeutic pulse laser system comprising:
a handheld master pulse laser;
a handheld secondary pulse laser having laser output;
a communication link between the master pulse laser and the secondary pulse laser providing for the downloading of a therapeutic protocol from the master pulse laser to the secondary pulse laser for remote execution by the secondary pulse laser; and
means for producing audio output at a select time during execution of the therapeutic protocol by the secondary pulse laser.

22. The therapeutic pulse laser system of claim 21 further comprising means for producing synthetic speech output at a select time during execution of the therapeutic protocol by the secondary pulse laser.

23. The therapeutic pulse laser system of claim 22 further comprising means for delivering device placement instructions from the secondary pulse laser to an operator by synthetic speech output.

24. The therapeutic pulse laser system of claim 23 further comprising means for delivering device operation instructions from the secondary pulse laser to an operator by synthetic speech output.

25. The therapeutic pulse laser system of claim 21 further comprising a harness for securing the therapeutic pulse laser in an operative position.
